(19) United States
(12) Patent Application Publication
(10) Pub. No.: US 2003/0008817 A1
(43) Pub. Date: Jan. 9, 2003

(54) Cross-link reversing agent

(76) Inventors: Tom Sander, Alachua, FL (US); Russell S. Donda, Alachua, FL (US)

Correspondence Address: VAN DYKE & ASSOCIATES, P.A., 7200 LAKE ELLENOR DRIVE, SUITE 252, ORLANDO, FL, 32809, US

(21) Appl. No.: 09/898,913

(22) Filed: Jul. 3, 2001

(51) Int. Cl. A61K 38/39; A61K 31/728; A61K 31/737; A61K 31/426

(52) U.S. Cl. 514/12; 514/54; 514/365

(57) ABSTRACT

The present invention generally relates to compositions and methods for restoring normal mechanical properties to collagenous tissue damaged as a result of the natural aging process in mammals. Accordingly, compositions are disclosed which comprises one or more thiazolium based agents capable of inhibiting and reversing non-enzymatic cross-linking of collagenous proteins, combined with one or more viscosupplement agents useful in replenishing structural and support material of a tissue or joint, which have been destroyed or damaged over time. The method comprises contacting the target tissue or joint capsule with the composition. Administration of the disclosed compounds can stop the progression of, or completely cure degenerative joint diseases.

### FIELD OF THE INVENTION

The present invention relates generally to the inhibition and reversal of time-dependant damage to collagenous tissue. Specifically, compositions and methods are disclosed directed to restoring normal biomechanical and biophysical properties to collagenous tissue damaged as a result of non-enzymatic cross-linking of proteins associated with the natural aging process in mammals.

### BACKGROUND

Age related changes in collagenous tissue manifested in the stiffening of joints, the deterioration of intervertebral discs, a decrease in the elasticity of the vascular system, and a decrease in permeability of renal and retinal capillaries. (Bailey et al, Mech Aging Dev December 1; 106(102):1-56) These changes are primarily due to non-enzymatic reactions between glucose and other reducing sugars with proteins, such as collagen, elastin, lens protein, kidney glomerulus basement membranes and others. (Maillard, L. D. C.R. Acad. Sci., (1912)154 pp.66-68) This “Maillard” or browning reaction produces short-lived “Amadori” products in vivo. These Amadori products are brown pigments, and have been found in long-lived proteins such as collagen, lens crystalline and nerve proteins. (Bucala et al Advances in Pharmacology, 1992 (23):1-34) Over time, Amadori products present in collagenous tissue slowly dehydrate and re-arrange to produce advanced glycosylation end-products (AGE's), that in turn promote cross-linking of proteins. Once cross-links are established, a matrix forms which traps other proteins. This process causes substantial and, heretofore considered irreversible, damage to protein structure over time. Cross-linking of collagen has been implicated in many problems in vivo because AGE's are involved in numerous biological processes relating to protein turnover. It is believed that their excessive accumulation leads to “protein aging” manifest in reduced skin elasticity, wrinkling, kidney disease, arthersclerosis, osteoarthristis, and many other age-related complications.

Damage to collagenous tissue associated with bone is particularly problematic because the cumulative affects over time may lead to a host of disabling diseases. Arthritis, for example, is among the most common chronic condition in the United States affecting approximately 40 million people at an annual cost of over $65 billion dollars (Summer, L, National Academy on Aging Society: Arthritis, 2000 March (5):1) Over one million surgical procedures in the United States each year involve cartilage replacement. (Graves, E. J. Advanced Data from Vital Health Statistics, Jun. 19, 1990 (185):1-12).

The surface of bones that articulate with one another is covered by cartilage, which prevents bone-to bone contact and allows near frictionless movement in joints. This tissue is composed primarily of proteoglycans and collagen fibers. Proteoglycans are known to play a role in the maintenance of hydration levels of articular cartilage as well as provide for elasticity and stiffness on compression. Collagen molecules provide for structural integrity through cross-linkage of fibers. One of the age-related changes in collagenous tissues is the reduction in proteoglycans, resulting in dry, brittle tissue unable to provide adequate buffering between bones. For example, the mechanical response of an intervertebral disc depends on its fluid content, which in-turn directly depends on the proteoglycan content of the nucleus. However, the molecular weight and concentration proteoglycan content in the disc nucleus decreases with age, leading to dehydration of the nucleus, and other deleterious changes which negatively impact on a discs mechanical properties (Urban J P ; McMullin J F., Spine, Feb. 13, 1998(2):179-87 As the disc nucleus dehydrates and shrinks with age, the load on the nucleus decreases and the load on the annulus increases. Radial tears, crack and fissure occur first within the annulus. If healing does not occur, the nucleus may migrate from the center of the disc to the periphery through the tear and possibly compress a nerve. As the nucleus pulposus dries out, its effectiveness as a “shock absorber” is reduced and lead to the formation of bone spurs that may compress nerves. If the natural cushioning capacity of a disc is lost, a space may develop between vertebrae causing nerve compression in the neuroforamen. Further, because the disc is avasucular and obtains its nutrients through diffusion, changes in structure of a disc significantly alters its ability to remain healthy. Another age-related change in collagenous tissue is the exponentional increase in senescent collagen cross-links. Changes in the cross-link profile of the disc indicate increased matrix turnover and tissue remodeling and may have implications for the progression of many dibilitating disorders. For example, cross-linking of collagenous tissue in a human intervertebral disc leads to a significant alteration in its mechanical ability to respond to normal stress. (Duance et al, Spine Dec. 1, 1998:23(23): 2545-51). Recent studies have demonstrated that non-enzymatic derived cross-linking increases with age in human intervertebral discs, and contributes to disc degradation. This problem can result in intense pain, often requiring surgical attention. U.S. Pat. No. 5,047,055 issued to Bao discloses a hydrogel nucleus implant designed to mimic the capacity of the nucleus tissue to absorb and release water under cyclic loading conditions. The implant contains 70% water content under physiological conditions and restores normal mechanical properties to damaged disks. However, this implant material does not solve the problem because proteins within the disc may continue to undergo advanced glycosylation and form cross-links. Thus, despite its replacement of the nucleus, the annulus fibrosus is still prone to age-related deterioration that in turn will affect the ability of the disc to respond to mechanical stress.

In articular cartilage of the knee, age-related cross-linking leads to embrittlement of tissue and consequential loss of “healthy” mechanical properties. (Takahashi et al Arthritis Rheum 1994 May: 37(5):724-728) Articular cartilage AGE levels have been correlated with a decrease in proteoglycan synthesis with age, and may be responsible for the age-related declines in the synthetic capacity of cartilage (DeGroot, J. et al., Arthritis Rheum 1999 May 42 (5):1003-9) With age, articular cartilage progressively loses its ability to self repair. Thus damage to this very sensitive and delicate layer by disease will leave a permanent effect on physiology and function of the knee. Painful inflammation of a joint may result as cartilage tissue that serves as a natural buffer between bone, becomes brittle and non-functional. Other studies have demonstrated that cross-linking of collagen proteins decreases the critical flexibility and permeability of the tissues, leading to a reduction in tissue replacement. (Paul, R G. And Bailey, A J., Int. J. Biochem Cell Bio. 1996 December: 28 (12):1297-310). Conversely, inhibition of cross-link formation in collagen has been associated with reduced stiffness in vessels. (Bruel et al., Artherosclerosis 1998 September:14(1):135-45)

Until recently, time-related damage to collagenous tissue was thought to be irreversible. As a result, therapies have focused on treating the symptoms of the disease, rather than the cause. For example, spinal fusion has been used where intervertebral discs have been permanently removed, but this often leads to increased stress and degeneration in adjacent discs as the body attempts to stabilize. Synthetic intervertebral discs have been used to supplant the mechanical function of a damaged disc, but these wear out over time and are incapable of regenerating or repairing cartilage. Other therapies have included chemonucleolysis, which involves injecting an enzyme into the nucleus to dissolve tissue that has been damaged, or that is placing pressure on a nerve. However, the removal of supporting tissue will ultimately cause the compressive load to be distributed to other sections of the disc, which in turn may cause further damage. Total joint replacements have been employed where articular cartilage has been extensively damaged, but these wear out over time and need replacement. Autologous grafting, and allograft implants have been used, but both require surgical procedures which are expensive and may not be permanent. Non-surgical alternatives have included changes in lifestyle, physiotherapy, and injection of non-steroidal anti-inflammatory drugs, in combination with various pain reducing medications. Intra-articular injections of steroids hormones such as, for example, corticosteroids have been used to reduce pain associated with inflammation. However, this type of therapy provides only temporary relief of pain, and may often lead to secondary complications such as degeneration of osteocartilage. More recently therapies have been directed at re-nourishing damaged tissue with structural or supporting materials that have been depleted over time.

Glycosaminoglycans (GAG's), such as, for example, hyaluronic acid, chondrotin sulfates and glucosamines, are a group of natural compounds that form an integral part of skin, cartilage, joint, organs, and other important tissues including many body fluids. They play a role in cartilage development and repair and may contribute to the function of healthy joints. The therapeutic potential of one GAG, hyaluronic acid (HA), has been studied with increasing interest recently. HA functions as the backbone of the proteoglycan aggregates necessary for the functional integrity of articular cartilage of the knee. (Leslie M., Nurse Pract., Jul. 24, 1999(7):38, 41-8) It is an important component of the extracellular matrix and is known to be involved in hydration, lubrication, and solute transport as well as providing elasticity to the joints and rigidity to intervertebral discs. It is a principal component of cartilage and helps with inflammation and discomfort associated with tissue damage. Notably, HA has an unsurpassed ability to retain water within the matrix. Recently, intra-articular hyaluronic acid injections have been used with some success to treat degenerative joint diseases, such as, for example, osteoarthritis. Several studies have demonstrated that repeated intra-articular injections of hyaluronic acid can alleviate pain and possibly even repair cartilage damage in early stage osteoarthritis. (Listrat et al., Osteoarthritis Cartilage, May 5, 1997(3):153-60) Intra-articular injections of HA were shown to control stages of the inflammation processes in synovial fluid, aid in reconstruction of the cartilage surface, help reduce proteoglycan depletion, and protect the viability of chondrocytes (Leardini Clin Ther 10, (5):521-6 1988). These affects are more pronounced in patients 60 years or older suffering from knee osteoarthritis. (Lohmander L S., et al Ann Rheum Dis., 1996 July,55 (7):424-31) Patients undergoing these injections have enjoyed protracted symptomatic remission periods, but never complete recovery. Thus, despite its promising therapeutic powers, HA does not have any pharmacological properties that would allow it act to cure the disease. Thus, hyaluronic acid injections solve only part of the problem associated with tissue damage.

Recently, a breakthrough in the understanding of cross-link mechanisms has lead to the identification of compounds capable of cleaving protein-protein cross-links, thus alleviating some of devastating affects of aging. U.S. Pat. No. 6,007,865 ('865) discloses a group of thiazolium based compounds which have the capacity to cleave dicarbonyl-based protein cross-links present in AGE's. Treatment with the disclosed compositions has wide ranging applications for areas of the body where collagenous tissue is present, such as, for example, joints, vessels, organs or other tissues. However, these compounds only solve part of the problem associated with degenerative diseases. None of the compounds disclosed in the '865 patent have the ability to restore normal, healthy function to structures such as articular cartilage, or intervertebral discs, which have been progressively damaged over many years. One reason for this may be found in the natural decreases observed in hyaluronic acid, proteoglycans, and other materials with age. Once pre-formed cross-links are cleaved in these tissues, a dehydrated, brittle matrix of unstable tissue remains. Thus, despite their therapeutic value, agents capable of inhibiting the formation of advanced glycosylation end-products, and reverse pre-formed AGE mediated molecular cross-links in the body do not completely solve the problem associated degenerative joint disease. Thus, a need remains in the field for a complete therapy to stop the progression of these diseases and restore normal function to collagenous tissue.

### SUMMARY OF THE INVENTION

The present invention provides compositions, and methods of their use, which are capable of breaking pre-formed AGE-related cross-links, inhibiting the formation of AGE's, and restoring normal mechanical properties to damaged collagenous tissue. The compositions comprise one or more primary agents capable of breaking advanced glycosylation mediated cross-links, combined with one or more secondary viscosupplement agents that function to replenish structural and support material normally found in healthy tissues and joints. Primary agents comprise therapeutic quantities of thiazolium compounds. Secondary agents comprise therapeutic quantities of hyaluronic acid, proteoglycans, collagen solution or other glycosaminoglycans. When combined, these two groups of agents are capable of preventing, arresting and reversing the progressive, age-related damage caused to structural body proteins as a result of advanced glycosylation and cross-linking in mammals. A method comprising contacting a target tissue with a composition according to the present invention is also disclosed. Administration of compositions of the present invention reduces, and often eliminates, the pathologies involving “protein aging”, such as osteoarthritis, peri-articular rigidity, loss of elasticity, wrinkling of skin, stiffening of joints, intervertebral disc degeneration, etc.

Accordingly, it is a principle object of the present invention to provide a composition capable of restoring normal mechanical properties to damaged collagenous tissue.

It is another object of the present invention to provide a method for restoring normal mechanical properties to damaged collagenous tissue.

It is a further object of the present invention to provide a composition capable of reversing the age-related deterioration of collagenous tissue.

It is a further object of the present invention to provide a method for reversing the age-related deterioration of collagenous tissue.

It is a further object of the present invention to provide a composition capable of reversing the age-related deterioration of collagenous tissue

It is still a further object of the present invention to provide a composition capable of inhibiting age-related deterioration of collagenous tissue.

It is still a further object of the present invention to provide a method for inhibiting age-related deterioration of collagenous tissue.

It is a further object of the present invention to provide a composition for the intra-articular treatment of damaged collagenous tissue in mammals.

It is a further object of the present invention to provide a method for the intra-articular treatment of damaged collagenous tissue in mammals.

It is a further object of the present invention to provide a composition for the intra-discal treatment of damaged intervertebral discs in mammals.

It is a further object of the present invention to provide a method for the intra-discal treatment of damaged intervertebral discs in mammals.

It is a further object of the present invention to provide a composition capable of curing degenerative disease of a mammalian joint.

It is a further object of the present invention to provide a method of curing degenerative degenerative disease of a mammalian joint.

Other objects and advantages will become apparent to those skilled in the art from a consideration of the ensuing description.

### DETAILED DESCRIPTION OF THE INVENTION

To date, individuals suffering from degenerative diseases of a joint have had few treatment options. Most therapies have focused on the symptoms of the disease rather than the cause, by administering analgesics, anti-inflammatory agents or combinations thereof. However, the inability to affect a significant change in the progression of the disease, often leads to painful complications that may ultimately lead to surgical intervention. It has now been unexpectedly discovered that degenerative joint conditions may be treated, repaired and in many cases completely eliminated through administration of a novel composition of primary and secondary agents disclosed in the present invention. Compositions, and methods of their use, are disclosed that are capable of inhibiting, arresting or reversing the affects of age-related damage to collagenous tissues. The compositions comprise therapeutic quantities of one or more primary agents capable of inhibiting the formation of, or breaking pre-formed, intermolecular protein cross-links, combined with therapeutic quantities of one or more secondary viscosupplementation agents. By primary agents is meant thiazolium, its pharmaceutically accepted salts, and mixtures thereof. Examples of these agents are disclosed in U.S. Pat. No. 6,007,865 issued to Alteon, which is incorporated herein by reference. By advanced glycosylation endproducts is meant those products resulting from the non-enzymatic reaction of glucose and proteins in vivo that have an ability to participate in protein-protein cross-linking.

By secondary viscosupplementation agents is meant those substances normally found in synovial fluid and collagenous tissue such as, for example, hyaluronic acid, collagen, proteoglycans, glycosaminoglycagens, etc. Glycosaminoglycans (GAGS) are mucopolysaccharides of animal origin. They are made up of residues of hexosamines glycosidically bound and alternating in a more-or-less regular manner with either hexuronic acid or hexose moietites. GAGs preferably make up at least about 5%, and more preferably, from about 6% to about 15% by weight of the polymer. Useful GAGs include those comprising sulfate groups such as chondroitin 4-sulfate, chondroitin 6-sulfate, hyaluronic acid, dermatan sulfate, keratan sulfate, heparin, heparan sulfate and combinations thereof. Preferably the matrix includes chondroitin 6-sulfate.

Together, these primary and secondary compounds affect numerous protein masses in the body, such as, for example, collagen, which deteriorates with age. By collagenous tissue is meant those tissues containing collagen that are found in association with amphiarthroidal and diarthroidal joints. Example of these include: hinge joints found in the elbows, knees, and fingers, pivot joints found between the first two vertebrae, intervertebral joints, gliding joints found between the various bones of the wrist and ankle, saddle joints found between the thumb and carpal bones, facet joints, sacroliliac joints, and others. Treatment for bursa, tendon and ligaments is also possible.

By repaired is meant to replace some or all of the normal structural or functional properties to damaged collagenous tissue. By degenerative joint disease is meant non-infectious, progressive disorders of the joints characterized by chronic degeneration of collagenous tissue, such as, for example, degeneration of the articulator cartilage, intervertebral discs, or other damage to cartilage covering bones that comprise an affected joint. The method involves contacting the damaged tissue with a composition according to the present invention.

In a preferred method, compositions are injected directly into the tissue, such as, for example, the intervertebral disc, or into the surrounding joint capsule, such as, for example, the synovial cavity. Damage to articular cartilage may be treated through intra-articular injections of compositions according to the present invention. Injection of the composition may be made by a syringe or cannula depending upon the consistency of the composition. In one embodiment, hyaluronic acid (HA) is combined with one or more thiazolium compounds in an injectable dosage unit. Hyaluronic acid can be a natural product made from rooster combs, such as, for example, Hyalagan® (Sodium Hyaluronate), Synvisc® Hylan (G-F 20) or Suplasyn®, or it may be obtained in synthetic form manufactured from bacterial cultures. Injection of this composition serves multiple purposes. The thiazolium compounds act to inhibit formation of new AGE mediated protein cross-links, while catalytically breaking existing cross-links in collagenous tissues. This increases flexibility of the tissue and breaks up protein masses trapped over time in the cross-link matrix. Viscosupplementation with hyaluronic acid helps to restore the properties of shock absorption and lubrication to diseased synovial fluid. Additionally, HA acid acts to reverse damage caused to cartilage by stimulating new production of chondrocytes. Further, by increasing the viscosity of the internal environment through this technique, pain and inflammation associated with contact between opposing surfaces of cartilage is eliminated. This treatment reduces the need for arthroplastic surgery on the knee to replace damaged cartilage. Patient undergoing this treatment experience a significant improvement in mobility, coupled with a reduction, or complete elimination of pain and inflammation. The composition has the potential to completely eliminate degenerative joint disease. However, specific results will vary with age of the patients, and stage of the disease. For example, in early to late stages where tissue has not been significantly destroyed, complete recovery is predicted. In advanced stages, where cartilage is significantly destroyed, treatment will stop the progression of the disease. Patients undergoing treatment at this stage may expect protracted symptomatic remission periods requiring follow up treatment. This creates an alternative for patients who may otherwise be forced to undergo surgery to minimize pain and inflammation.

Intervertebral discs damaged from the natural aging process may also be treated through intra-discal injections of compositions according to the present invention. In a preferred composition, high molecular weight proteoglycans are added to one or more thiazolium compounds. Generally, compositions will be directly injected into the nucleus puplosus. In situations where the viscosity of the material precludes use of a syringe, other devices such as a cannula may be employed. A method and apparatus for injecting material into a spinal disc quickly, and safely is disclosed in U.S. Pat. No. 6,206,921 issued to Guagliano, and is incorporated herein by reference. Injection of the present composition serves multiple purposes. The thiazolium compounds act to inhibit formation of new AGE mediated protein cross-links, while catalytically breaking existing cross-links. Breaking of these cross-link increases flexibility of the tissue and leads to a significant improvement in a discs mechanical ability to respond to normal stress. High molecular weight proteoglycans increase hydration of the tissue, which in turn promotes increased mechanical response of the disc. Following treatment, a disc is able to support a normal load, thus decreasing stress placed on the annulus. When completely re-hydrated in this way, nucleus is prevented from migrating from the center of the disc to the periphery, thus minimizing the potential for disc rupture. Additionally, the discs' effectiveness as a “shock absorber” is restored, thereby reducing the formation of bone spurs that may compress nerves and cause substantial pain. Because the disc is avasucular, and obtains its nutrients through diffusion, other nutritional supplements may be added to the mixture to promote healthy of the disc. By restoring the mechanical function of the disc, many of the complications associated with aging may be alleviated without resorting to dangerous surgeries, such as, for example, spinal fusion.

The progression of degenerative diseases such as, for example, osteoarthritis of the knee is prevented through administration of a compound according to the present invention. In a preferred method, a patient is first diagnosed with osteoarthritis through X-ray, MRI or other techniques known in the field. The skin covering the afflicted knee is then cleansed with a bacteriocide or other agent to minimize infection. A therapeutic quantity of a composition containing one or more primary agents and one or more secondary agents is then drawn into a sterile syringe, cannula or other device depending on the consistency of the material. The device is then inserted intra-articularly and the composition is slowly discharged into the cavity surrounding the bones, or directly into the collagenous tissue. The site of injection will vary, but is preferably made in the intercondylar notch, on either side of the patella. The device is then removed from the joint. The skin covering the knee is then cleansed once more. To diffuse the material throughout the joint, it is preferable that the joint be rotated through a range of motions following the procedure. Depending on the severity of the condition, one or more of these injections may be required. It is expected that individuals experiencing late stage osteoarthritis would likely require multiple injections over a given time period to regain normal function of remaining tissue. When administered, the composition is capable of restoring healthy mechanical properties to collagenous tissues of the knee damaged by natural aging processes. The primary agents are believed to function by cleaving -dicarbonyl bonds present in AGE-mediated protein cross-links, thereby disrupting pre-formed cross-links and restoring flexibility to the tissue surrounding the knee joint. Primary compositions are also believed to be capable of inhibiting the formation of advanced glycosylation endproducts by interacting with initially glycosylated molecules at an early stage in the glycosylation process, thereby preventing later formation of AGE's. The secondary agents replenish materials that have been destroyed, or degraded over time, thereby restoring the natural environment of the knee joint. By reversing age-related cross-links in cartilage, normal mechanical properties of a joint are restored, thus eliminating pain associated with loss of function and mobility.

Inevitable complications associated with aging are now eliminated or reduced through administration of the disclosed compounds. Patients experience an expanded range of motion with minimal or no pain, which in turn improves the quality of life. The composition is preferably in the form of an injectable dosage unit for treatment of osteoarthritis or other degenerative disease. Compositions containing therapeutic quantities of primary and secondary agents may be injected intra-articularly or intra-discally. However, in some situations the composition need not be introduced directly at the site or location of the affected tissue and may be administered at a remote site. For example, interphalangeal, metacarpophalangeal, and metatarsophalgeal tissues may be better served through topical administration of a material. Thus, compositions prepared in the form of an ointment or a cream are also contemplated. Similarly, joints covered by substantial tissue, such as, for example, ball and socket joints of the shoulder or hip may be better served through oral administration of a composition. Thus, capsules, pills, liquids or other forms of a composition for oral administration are also contemplated. The composition are sterilized by autoclaving or other methods well known in the art, prior to injection. It is preferred that viscosupplement agents, such as hyaluronic acid, proteoglycans, collagen and others are substanitally pure. The treatment regimen involves administration of material through injection or other method over a selected time frame to inhibit advanced glycosylation mediated cross-links and break those that have previously formed.

A novel method of evaluating the ability of thiazolium compounds to inhibit crosslinking and break existing cross-linkages is described in U.S. Pat. No. 6,007,865, and is incorporated herein by reference. A method of assessing improvement after treatment of degenerative joint afflictions is disclosed in U.S. Pat. No. 3,966,908 and is incorporated herein by reference. A method for identifying and measuring the extent of a pathological condition due to an accumulation of advanced glycosylation endproducts in an animal is also described in U.S. Pat. No. 6,007,865, and is incorporated herein.

In patients having advanced stage of a disease, characterized by near total destruction of tissue, implants may be indicated. The compositions of the present invention may be implanted in a carrier as an autograft or allograft. For example, hyaluronic acid, proteoglycans, collagen and other GAG's, as well as FGF and other growth factors may be imbedded in a matrix for implantation into a joint. Chondrocytes may also be infused into the matrix. Following implantation, pain and inflammation are reduced, followed by a re-growth of cartilagenous tissue. A biological carrier may be formed from carbohydrates, proteins, lipids, synthetic bio-compatable polymers, or mixtures thereof. Through use of this method guided tissue repair for conditions involving damaged collagenous tissue may be affected.

The following examples are illustrative of the invention and are not meant to be construed as limiting:

### EXAMPLE 1

### Efficacy of Combining Cross-Link Reversing Agent with Viscosupplementation Agents for Treatment of Knee Osteoarthritis

Patients suffering from the same degree of knee osteoarthritis as evidenced by X-ray and MRI analysis, are entered into a study. Subjects are randomly assigned to one of four experimental groups. A baseline range of motion and pain analyses is conducted on each subject. Each individual is then subjected to five weekly intra-articular injections of varying compounds. Individuals in Group 1 are injected with 16 mg Synvisc®. Individuals in Group 2 are injected with 16 mg of Synvisc® combined with 1 mg/kg of thiazolium salt. Individuals in Group 3 are injected with 1 mg/kg of thiazolium salt. Individuals in Group 4 are injected with a sugar placebo composition. Results are evaluated weekly according to pain, function, motion, and activity.

At the end of the trial period, each patient undergoes range of motion and pain analyses. Data obtained is compared to that recorded in the original baseline analyses. X-ray and MRI images indicate changes in the joint capsule and tissue of groups 1,2 and 3.as expected. Group two shows a marked improvement over groups 1 and 3. On all measures of treatment effectiveness, including joint mobility, pain, and range of motion, the patients in group 2 exhibit rapid and significant improvement. Group 1 patients demonstrate increased mobility and reduction in pain. Group 3 patients exhibit greater flexibility, but an insubstantial reduction in pain. Group 4 patients show no significant change. The combination of both agents is tolerated well by group two patients and no local or systemic adverse reaction is present. Results from this study will demonstrate that compositions according to the present invention are effective in treating damaged tissue and restoring normal function to a knee joint.

### EXAMPLE 2

### Efficacy of Combining Cross-Link Reversing Agent with Viscosupplementation Agents for Treatment of an Intervertebral Disc

Patients suffering from the same degree of disc degeneration as evidenced by MRI analysis are entered into a study. Subjects are randomly assigned to one of four experimental groups. Each subject undergoes baseline compression load testing to evaluate vertical loading on the nucleus pulposus and annulus fibrosus. Each individual is subjected to five weekly intra-discal injections of varying compounds. Individuals in Group 1 are injected with a composition containing proteoglycans. Individuals in Group 2 are injected with a composition containing a mixture of proteoglycans and 1 mg/kg thiazolium salts. Individuals in Group 3 are injected with of 1 mg/kg thiazolium salt in an aqueous solution. Individuals in Group 4 are injected with an aqueous sugar placebo composition.

At the end of the trial compression load testing is repeated to evaluate vertical loading on nucleus pulposus and annulus fibrosus. Groups 2 exhibits a marked increase in the ability of the nucleus pulposus to respond to mechanical stress. The nucleus pulposus of Group 2 discs show a greater ability to absorb and respond to the compressive load than any other group. Group 1 patients exhibit, a moderate change in disc compression with the annulus continuing to absorb most of the compressive load. Groups 3 and 4 exhibit no change. However, individuals in groups 2 and 3 show a marked improvement in flexibility. Results from this study will further demonstrate that the compressive load bearing capacity of the nucleus pulposus in moderately degenerated discs may be increased with the administration of proteoglycan compositions. Additionally, administration of thiazolium salts into the disc improves flexibility. The combination of both agents are tolerated well by group two patients and no local or systemic adverse reactions is present. Results will show that compositions according to the present invention are affective in treating damaged tissue and restoring normal function to an intervertebral disc. Administration of these compounds to damaged intervertebral discs restores the natural function of the nucleus pulposus and shifts the vertical load on the annulus fibrosus resulting from deterioration of the disk, back to the nucleus pulposus. Biomechanical studies will confirm restoration of disc anatomy and function after injection of composition according to the present invention.

The disclosure of all patents and publications cited in this application are incorporated by reference in their entirety to the extent that their teachings are not inconsistent with the teachings herein. It should be understood that the examples and embodiments described herein are for illustrative purposes only and that various modifications or changes in light thereof will be suggested to persons skilled in the art and are to be included within the spirit and purview of this application and the scope of the appended claims.

What is claimed is:

1. A composition capable of restoring normal mechanical properties to collagenous tissue damaged through natural aging process, comprising
a. at least one primary agent capable of cleaving advanced glycosylation end-product (AGE) mediated cross-links formed in collagenous tissue; and
b. at least one secondary viscosupplement renourishing agents.

2. The composition of claim 1, wherein said at least one primary agent is thiazolium, pharmaceutically acceptable salts thereof, or combinations thereof.

3. The composition of claim 1, wherein said at least one secondary agent is selected from the group consisting of hyaluronic acid, collagen solution, chondroitan sulfate, proteoglycans, or other glycosaminoglycans, and mixtures thereof.

4. The composition of claim 1, wherein said at least one primary agent is capable of cleaving α-dicarbonyl based protein cross-links in collagenous tissue resulting from the formation of advanced glycosylation end-products.

5. The composition of claim 1, wherein said at least one secondary agent is hyaluronic acid.

6. The composition of claim 1, wherein said at least one secondary agent increases lubrication within a joint, and renourishing said tissue with structural and support materials damaged or destroyed a result of the aging process.

7. A method for restoring normal, healthy mechanical properties to collagenous tissue damaged through the natural aging process, comprising contacting said tissue with a pharmaceutical composition containing at least one primary agent capable of breaking pre-formed, AGE mediated protein cross-links, and at least one secondary agent capable of renourishing said tissue with structural and support materials damaged or destroyed as a result of the aging process.

8. The method of claim 7, wherein said primary agent is selected from the group consisting of thiazolium, its pharmaceutically acceptable salts, and mixtures thereof.

9. The method of claim 7, wherein said at least one secondary agent is a glycosaminoglycan.

10. The method of claim 7, wherein said at least one secondary agent is hyaluronic acid, collagen solution, chondroitan sulfate, or proteoglycans, or combinations thereof.

11. The method of claim 7, wherein said composition is capable of improving normal, healthy mechanical properties to collagenous tissue found between amphiarthroidal and diarthroidal joints.

12. The method of claim 7, wherein said composition is injected intra-articularly.

13. The method of claim 7, wherein said composition is injected intra-discally.

14. A composition for preventing and inhibiting age-related deterioration of collagenous tissue, comprising:
a. a therapeutic quantity of at least one primary agent capable of reacting with an early glycosyslation product formed by the initial glycosylation of a collagenous protein in vivo
b. a therapeutic quantity of at least one secondary agent capable of replenishing structural and support materials damaged or destroyed through the natural aging process.

15. The composition of claim 14, wherein said primary agent is thiazolium, its pharmaceutically acceptable salts, or mixtures thereof.

16. The composition of claim 15, wherein said secondary agent is selected from the group glycosaminoglycans consisting of hyaluronic acid, collagen solution, chondroitan sulfate, proteoglycans, and combinations thereof.

17. The composition of claim 14, adapted to reverse complications of aging caused by the accumulation of advanced glycosylation endproducts, and depletion of natural lubricants and support material within a mammalian joint.

18. A method for treating a mammal to inhibit age-related damage to collagenous tissue comprising administering to said mammal an effective amount of a pharmaceutical composition, said pharmaceutical composition comprising a primary agent capable of inhibiting the formation of protein cross-links by reacting with an early glycosylation product formed by the initial glycosylation of collagenous protein, and a secondary agent capable of replenishing lost structural and lubricating components of a joint.

19. A method for preventing, inhibiting and reversing affects of degenerative joint afflictions in mammals comprising:
a. injecting a therapeutically effective dosage of the composition of claim 1 directly into the tissue or joint; and
b. moving the joint through a range of motions to diffuse the injected material.

20. A method for curing a joint undergoing progressive cartilage degeneration caused by proteoglycan degradation, comprising administering intra-articularly to said joint an amount of a composition comprising a primary agent capable of breaking pre-formed AGE mediated cross-links, and a secondary agent containing a therapeutic quantity of proteoglycans.

21. A method for preventing steroid arthropathy of a mammalian joint, comprising administering intra-articularly to said joint a composition comprising at least one primary agent capable of inhibiting the formation of AGE mediated cross-links, and an effective amount of at least one secondary agent, wherein said at least one secondary agent is hyaluronic acid, or proteoglycans, or an admixture of both.

22. An improved method of treating a damaged intervertebral disc wherein the improvement comprises injecting a composition into said intervertebral disc; said composition comprising a primary agent capable of cleaving pre-formed protein crosslinks, and a secondary agent capable of replenishing glycosaminoglycogen content of said disc

23. The composition of claim 1, wherein said composition is provided in a pharmaceutically acceptable carrier for implantation.

24. The composition of claim 23, wherein said carrier is selected from the group consisting of carbohydrates, proteins, lipids, synthetic polypeptides, biocompatible natural and synthetic resins, antigens, and mixtures thereof.

25. A method of treating arthritis comprising administering to a patient in need thereof, an amount of the composition of claim 1, wherein said composition is administered parenterally, topically, or orally.
